(19) United States
(12) Patent Application Publication
(10) Pub. No.: US 2006/0257380 A1
(43) Pub. Date: Nov. 16, 2006

(54) Use of sirnas for gene silencing in antigen presenting cells

(75) Inventors: Anne Galy, Fontainebleau (FR); Diego Laderach, Corbeil-Essonne (FR); Daniel Compagno, Corbeil-Essone (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignees: Inst.Nat. De La Sante Et De La Recherche MED, Paris (FR); Genethon, Evry (FR); Institut Gustave Roussy, Villejuif (FR)

(21) Appl. No.: 10/528,569

(22) PCT Filed: Sep. 19, 2002
(86) PCT No.: PCT/EP02/12636

§ 371 Date: Oct. 5, 2005

(51) Int. Cl. A61K 48/00; C12N 15/09

(52) U.S. Cl. 424/93.21; 435/455

(57) ABSTRACT

The present invention relates to the use of small interfering RNAs (siRNAs) for silencing gene expression in antigen-presenting cells such as dendritic cells, in particular for immunomodulatory purposes.

The present invention relates to the use of small interfering RNAs (siRNAs) for silencing gene expression in antigen-presenting cells such as dendritic cells, in particular for immunomodulatory purposes.

RNA interference (RNAi) is a mechanism involving double-stranded RNA (dsRNA) molecules and resulting in post-transcriptional sequence-specific silencing of gene expression.

It is a multistep process, involving in a first step the cleavage, through the action of the Dicer enzyme (a RNase III endonuclease), of large dsRNAs into 21-23 ribonucleotides-long double stranded effector molecules called small interfering RNAs (siRNAs). These siRNAs duplexes bind to a protein complex to form the RNA-induced silencing complex (RISC). The RISC specifically recognises and cleaves the endogenous mRNAs containing a sequence complementary to one of the siRNA strands.

This mechanism was initially described in plants, worms, drosophila and parasites, where dsRNAs have been successfully used to induce gene-specific post transcriptional silencing.

However, in upper animals, such as vertebrates and in particular mammals, large dsRNAs (longer than 30 bp) elicit a type I interferon response predominantly leading to the activation of protein kinase R (PKR) (WILLIAMS, Oncogene 18, 6112-6120, 1999). In many cell types, this results generally in a nonspecific degradation of RNA transcripts and a general shutdown of translation.

This obstacle to the use of RNA interference for gene specific silencing in mammals has been recently overcome by the use of siRNAs (TUSCHL et al., Genes Dev. 13, 3191-3197, 1999; ELBASHIR et al., Nature 411, 494-498, 2001). By way of example, siRNAs consisting of 19-25, preferably 19-23 nucleotides, with overhanging 3′-ends are described in PCT WO 02/44321.

Due to their small size, the siRNAs fail to activate the PKR pathway, and it has been shown that they were able to induce a specific and strong reduction of protein expression in cultures of fibroblast and epithelial cell lines (HARBORTH et al., J. Cell. Sci. 114, 4557-4565, 2001), and of primary lymphocytes (JACQUE et al., Nature 418, 435-438, 2002) as well as in vivo in mice (McCAFFREY et al., Nature, 418, 38-39, 2002).

Antigen presenting cells (APC) constitute a complex system of cells that capture, process and present antigens to lymphocytes and play prominent roles in infectious diseases, cancer, immune disorders and vaccination. APCs include monocytes/macrophages, B lymphocytes, dendritic cells (DC); the most potent APCs being DC. The DC system consists of a complex system of cells that are uniquely capable of activating naive T lymphocytes thus, unlike other APCs, can initiate immune responses. A well-characterized type of DC is the monocyte-derived DC that is produced in vitro by culture of human blood monocytes.

There is great interest in understanding mechanisms of DC activation. DC integrate a variety of signals from pathogens, inflammatory mediators or T cells that condition their ability to present antigen to naive T cells and to subsequently regulate the development of immune responses (LANZAVECCHIA et al., Cell 106, 263-266, 2001 MELLMAN et al., Cell 106, 255-258, 2001). One can recognize three major categories of signals that regulate the function and activation of DC. The first relates to the recognition and processing of pathogens or antigen-associated motifs. Bacterial and viral constituents such as lipopolysaccharides (LPS), dsRNA, CpG motifs of bacterial DNA are recognized by specialized Toll-like receptors (TLR) on DC and trigger cytokine production and cellular activation of DC. Another influence on DC is the environmental milieu for instance cytokines, chemokines, hormones or small molecules that have pro- or anti-inflammatory activity and are produced during innate or adaptive immune responses. Notably, interleukins (IL) like IL-1 or IL-4 modulate the differentiation of DC and their response to other activation signals. A third type of signal involves receptors and ligands engaged by cognate cell-to-cell interactions. Examples include interactions between DC and T lymphocytes via molecules of the tumor necrosis factor (TNF) receptor/ligand superfamilies that are prominent regulators of DC activation, survival and differentiation. For example, CD40 ligand (CD40L), induces the maturation of DC in vitro, enhancing their ability to interact with naive T cells through up-regulation of MHC class II and co-stimulatory antigens on the cell surface. Further, CD40L in conjunction with mediators of innate immunity such as IL-1, induces the transcription of IL-12α and β mRNA and the production of high levels of the heterodimer interleukin-12 (IL-12)αβ in DC (WESA & GALY, Int. Immunol., 2001, August; 13, 1053-61; LUFT et al., J. Immunol. 168, 713-722, 2002). The cytokine IL-12 is a deterministic factor for the development of cellular immunity, inducing Th1 T cell differentiation and the production of high levels of IFN-γ by T and Natural Killer (NK) lymphocytes (TRINCHIERI et al., Curr. Top. Microbiol. Immunol. 238, 57-78, 1999).

Thus, it appears that the molecular mechanisms that regulate DC activation and the production of cytokines by DC are pivotal events that control the development of cellular immune responses.

The transduction of signals from TNF receptor superfamily and the interleukin-1 receptor/Toll-like receptor (IL-1R/TLR) superfamily is mediated by TNF receptor associated factors (TRAFs). To date, six members of this family of homologous proteins have been described. TRAF proteins are important regulators of cell death, cellular responses to stress and TRAF2, TRAF5 and TRAF6 have been reported to mediate activation of NF-kappaB and jun kinase. In DC, TRAF-3 is recruited in membrane rafts by engagement of CD40 on the surface of the DC (VIDALAIN et al., EMBO J. 19, 3304-3313, 2000). Thus potentially, TRAF-3 plays an important role in the response of DC to this mode of activation but a role for TRAF-3 in DC has not been clearly established. Mice rendered genetically null for TRAF3 die rapidly and fail to develop a competent immune system (XU et al., Immunity 5(5), 407-415, 1996).

In DC, pro-inflammatory signals of innate or adaptive immune responses generally lead to the activation of NF kappa B/Rel for the transcription of target genes. In mammalian cells, NE kappa B/Rel proteins consist of p50 (NF-κB1), p52 (NF-κB2), p65 (RelA), RelB, c-Rel that are encoded by different genes and play non-redundant roles of importance in various aspects of development, inflammation and immunity (BURKLY et al., Nature 373, 531-536, 1995; FRANZOSO et al., J. Exp. Med. 187, 147-159, 1998). NF kappa B/Rel proteins form homo- or hetero-dimers maintained in the cytosol by association to inhibitory IKB proteins. A variety of inflammatory, pathogen-derived, stress or developmental stimuli, transmitted by the pathways mentioned above, activate the IKB kinase complex, subsequently triggering the phosphorylation of IKB and its degradation in the proteasome. This releases p50 or p52 that form, with Rel proteins, heterodimers that are translocated to the nucleus and activate the transcription of target genes (GHOSH et al., Annu. Rev. Immunol. 16, 225-260, 1998). Further phosphorylation events regulate the activity of Rel proteins in the nucleus. In addition, homodimers of p50 or p52 exist that acquire transactivating potential by binding to Bcl-3, a member of the IKB family of proteins.

A major role of NF kappa B/Rel proteins in antigen presentation has been first suggested by localization studies in tissues or in cells then by the phenotype of animals with targeted mutations. Individually, p50, IκB-α, c-Rel, RelB, p65, Bcl-3 and p52 knockout mice have been produced with impairment of several immunologic parameters (reviewed in SHA, J. Exp. Med. 187, 143-146, 1998). OUAAZ et al. (Immunity. 16, 257-270, 2002) report that development and function of murine BM-derived DC were not affected by lack of individual NF kappa B subunits, while on the other hand the combined absence of p50 and Rel-A abrogates the formation of all subsets of DC; the lack of p50 and c-Rel together strongly reduced IL-12 production but had no significant effect on expression of MHC and costimulatory molecules. In human cells, differential expression of NF kappa B/Rel genes is found during the in vitro differentiation of monocytes into DC or macrophages and complexes consisting of p50, RelB and c-Rel are found in the nucleus of mature monocyte-derived DC (RESCIGNO et al., J. Exp. Med. 1188, 2175-2180, 1998; NEUMANN M et al., Blood. 95, 277-285, 2000). Transfection of RelB cDNA in B cell lines increases expression of MHC class I and CD40 cell surface expression and enhances MHC class I-peptide-mediated activation of CD8⁺ T cells (O'SULLIVAN et al., Proc Natl Acad Sci USA. 97, 11421-11426, 2000). Thus, NF kappaB/Rel proteins are associated with the development of the antigen-presenting cell system as their expression correlates with the activation of various types of APCs and with the differentiation of non-professional APCs such as monocytes/macrophages into professional APCs like dendritic cells. However, it is unclear how individual constituents of NF kappa B regulate the activation of human DC.

As a viral constituent, dsRNA is recognized by APCs as a pathogen-associated motif that leads to cellular activation. Thus, dendritic cells react to stimulation with dsRNA in a quite different way than other cell types: in contrast to other cells where dsRNA induces via the activation of PKR a general shutdown of translation, dendritic cells respond to dsRNA by an increase in protein synthesis, and up-regulation of MHC and co-stimulatory antigens, allowing a high level of production and presentation of viral antigens (CELLA et al., J. Exp. Med. 89(5), 821-829, 1999). It has been reported (ALEXOPOULOU et al., Nature 18, 413, 732-738, 2001) that DC specifically recognize dsRNA via Toll-like receptors, in particular Toll-like receptor 3 (TLR3); activation of this receptor induces the activation of NF-κB and the production of type I interferons. Messenger RNA for TLR3 has been found in immature and mature monocyte-derived DC but its presence in monocytes is controversial (VISINTIN et al., J. Immunol. 166, 249-255, 2001; KADOWAKI et al., J. Exp. Med. 17, 194(6), 863-869, 2001). Collectively, the expression of TLR is not restricted to antigen-presenting cells but is found also on leukocytes and fibroblasts. However, only DC express the full repertoire of TLR, in particular, DC are the only leukocytes that express TLR3, the putative receptor for dsRNA. (MUZIO et al., J. Immunol. 164, 5998-6004, 2000). It has been shown that binding of dsRNA to DC or to TLR3-transfected epithelial cells induces an IFN response (KADOWAKI et al., precited; MATSUMOTO et al., Biochem. Biophys. Res. Commun., 293, 1364-1369, 2002). These results suggest that the uptake of, and response to, dsRNA may be distinct in DC expressing TLR3 such as monocyte-derived DC, compared to other types of cells: these results also suggest that the potential activation of TLR3 by siRNA could cause non-specific IFN response, mortality and translation shut-down, thus preventing the effective use of siRNA in DC.

In view of the above, the functionality of RNA interference in APC was uncertain, since a mechanism resulting in elimination of viral RNA would result in a decrease in the production of viral antigens, and thus in a less efficient presentation thereof.

The inventors have tested if siRNAs were able to induce in dendritic cells either a non-specific type-I interferon response or a gene specific silencing.

They have found that double stranded RNA molecules of 21-23 ribonucleotides did not elicit any non-specific type-I interferon response. In contrast, they found that a strong gene specific silencing was elicited when these RNA molecules were siRNAs directed against genes expressed in dendritic cells.

In particular, they found that the transfection of dendritic cells with siRNA directed against the p50 gene induced a specific decrease of p50 expression. In contrast with the observations previously reported by OUAAZ et al., they found that this reduction of p50 expression was sufficient to induce a strong reduction of secretion of IL-12, and that co-transfection of DC with siRNA directed against the p50 gene and siRNA directed against the c-Rel gene further induced a significant reduction of the expression of MHC and costimulatory molecules.

In addition, they found that transfection of dendritic cells with siRNA directed against the gene encoding TNF-receptor associated factor 3 induced a strong reduction of secretion of IL-12.

Further, they also found that DC transfected with siRNA directed against genes encoding either p50 or TRAF3 failed to activate the production of IFN-gamma by T lymphocytes.

The invention thus provides new means for modulating the immune response, through siRNA mediated gene silencing in dendritic cells, more specifically human dendritic cells. In particular, the invention provides means for decreasing IL-12 production by dendritic cells. The invention also provides means for suppressing an unwanted Th1 T cell response.

The present invention thus relates to the use of siRNAs to down-regulate the expression of one or more target(s) gene(s) in an antigen presenting cell, in particular a dendritic cell or a precursor thereof, and preferably a monocyte-derived dendritic cell or a precursor thereof. Advantageously, said antigen presenting cell is a human cell.

In particular, an object of the invention is a method for obtaining isolated or cultured antigen presenting cells wherein the expression of one or more target(s) gene(s) is down-regulated, wherein said method comprises introducing in said cells siRNA(s) directed against said target(s) gene(s).

From the sequence of a chosen target gene, one of skill in the art can easily design and prepare siRNA directed against said target gene, by means known in themselves, as disclosed for instance by ELBASHIR et al., (Nature, 2001, cited above; EMBO J. 20, 6877-6888, 2001) or in PCT WO 02/44321. Introduction of said siRNA in the cells can be performed either by direct transfection, for instance by electroporation or liposome mediated transfection, or by means of an expression vector comprising a DNA template for the chosen siRNA placed under transcriptional control of a polIII promoter. A DNA template for siRNA comprises the DNA sequences to be transcribed into the sense and antisense strands constituting the siRNA duplex. At the present time, two kinds of expression vectors for siRNA have been proposed (TUSCHL, Nature Biotechnol., 20, 446-448, 2002). In the first one, the sense and antisense sequences of the DNA template are placed in separate transcription units (LEE et al., Nat. Biotechnol. 20, 500-505, 2002; MIYAGISHI & TAIRA, Nat. Biotechnol., 20, 497-500, 2002). In the second one, a single promoter controls the expression of the sense and antisense sequences of the DNA template, that are separated by a short spacer region; the transcription of this construct results in small-hairpin RNA (shRNA) that give rise to siRNA after intracellular processing involving the enzyme Dicer (McCAFFREY et al., Nature, 2002, cited above; BRUMMELKAMP et al., Science, 296, 550-553, 2002; PADDISON et al., Genes Dev. 16, 948-958, 2002).

A particular embodiment of the invention includes the selection of a target gene among:
a gene encoding the p50 subunit of NF-κB;
a gene encoding TNF-receptor associated factor 3;
a gene encoding the c-Rel subunit of NF-κB.

Another embodiment of the invention includes the selection of a target gene encoding the p50 subunit of NF-κB and a target gene encoding the c-Rel subunit of NF-κB.

The invention also encompasses siRNA directed against a target gene selected among:
a gene encoding the p50 subunit of NF-κB;
a gene encoding TNF-receptor associated factor 3;
a gene encoding the c-Rel subunit of NF-κB; as well as expression vectors comprising a DNA template for said siRNA.

Expression vectors of the invention include gene therapy vectors, in particular gene therapy vectors derived from viruses such as Murine Moloney Leukemia virus, Human immunodeficiency virus (HIV-1), Simian immunodeficiency virus (SIV), foamy virus, adeno-associated virus, adenovirus, canine adenovirus, canarypox virus, herpes virus. Preferred virus-derived vectors for antigen presenting cells, including dendritic cells, are derived from Murine Moloney Leukemia virus, HIV, SIV, or adenovirus.

Another object of the invention is the use of siRNAs or expression vectors of the invention as medicaments.

According to a preferred embodiment of the invention, siRNA directed against a target gene selected among:
a gene encoding the p50 subunit of NF-κB;
a gene encoding TNF-receptor associated factor 3;
a gene encoding the c-Rel subunit of NF-κB;

or a vector expressing said siRNA is used for preparing a therapeutic composition, in particular an immunosuppressive composition, for treating or preventing a disease resulting from an overproduction of IL-12 by dendritic cells.

Diseases resulting from an overproduction of IL-12 by dendritic cells include for instance pathologic conditions in which adaptive responses are elicited against self-antigens, such as autoimmune diseases ranging from systemic to organ specific such as systemic lupus erythematosus, rheumatoid arthritis, multiple sclerosis, insulin-dependent diabetes mellitus, Hashimoto's thyroiditis, myasthenia gravis.

An overproduction of IL-12 is also implied in adverse immune response against the graft in tissue or organ transplantation, or against vectors used to correct genetic deficiencies in gene transfer therapies. Accordingly, the siRNAs of the invention, or the corresponding expression vectors, can also be used in the treatment of diseases resulting from said immune response. In some cases, one may wish to obtain a more drastic immunosuppressive effect: this can be done by reducing at once the production of IL-12 and the expression of MHC and costimulatory molecules, by use of a combination of siRNA directed against a target gene encoding the p50 subunit of NF-κB, with siRNA directed against a target gene encoding the c-Rel subunit of NF-κB, or of the corresponding expression vectors.

The present invention also provides antigen presenting cells, in particular dendritic cells or precursors thereof, obtained by the method of the invention. These antigen presenting cells contain siRNA(s) directed against target gene(s) expressed in said dendritic cell.

The invention further provides pharmaceutical compositions comprising antigen presenting cells of the invention. The invention also provides pharmaceutical compositions comprising T lymphocytes and dendritic cells.

The present invention also provides a method to produce T lymphocytes that fail to produce IFN-gamma, wherein said method comprises inducing the activation of naïve T cells by co-cultivating said T cells with an antigen presenting cells of the invention, containing siRNA directed against a gene encoding p50 or TRAF-3.

The present invention will be further illustrated by the additional description which follows, which refers to examples demonstrating the effect of siRNAs in dendritic cells. It should be understood however that these examples are given only by way of illustration of the invention and do not constitute in any way a limitation thereof.

### EXAMPLE 1

### EFFECT OF SIRNA TARGETING NF KAPPA B P50 AND C-REL IN DENDRITIC CELLS

siRNAs

21-nucleotide double-stranded RNA with two overhangs dT nucleotides, targeting NFκB p50 (GGG GCU AUA AUC CUG GAC UdTdT; SEQ ID NO:1), and cRel (CAA CCG AAC AUA CCC UUC U dTdT; SEQ ID NO:2) were designed from the sequences of the corresponding genes. Control double-stranded RNAs having randomly scrambled sequences (scramble I: UGU UUU AAG GGC CCC CCG UdTdT; SEQ ID NO:3, scramble II: CGG CAG CUA GCG ACG CCA UdTdT; SEQ ID NO:4) were also prepared.

The sequences indicated above are the sense sequences of the siRNAs. The sequence for p50 as well as the sequence for cRel failed to reveal significant sequence homologies with other known genes (including other members of the same families) after standard BLAST search. Similarly, control scramble RNAs failed to reveal significant sequence homologies with any known genes after standard BLAST search.

Dendritic Cells

Mononuclear cells (MNC) were isolated by centrifugation over Ficoll (Amersham Pharmacia Biotech, Piscataway, N.J.) (d<1.077 g/ml) from cord blood samples and were cryopreserved in liquid nitrogen using a 10% DMSO freezing solution.

Monocytes were obtained by incubating MNC on tissue culture plates (2×10⁶ cells per ml per well in 24 well plates) in RPMI medium with 10% fetal bovine serum (FBS) (R10) ²⁷ in a humidified atmosphere at 37° C., 5% CO₂ for 2 hours, followed by washing to remove non-adherent cells. These adherent cells were cultured in R10 medium with GM-CSF (25 ng/ml, Immunex, Seattle, Wash.), and IL-4 (10 ng/ml, RD Systems, Minneapolis, Minn.) for 4 to 6 days to induce DC differentiation.

These immature human monocyte-derived DC cells were transfected by electroporation with various concentrations of p50 or control siRNAs.

Transfection of siRNAs

Transfection of siRNAs was carried out by electroporation with a square wave electroporation system (BTX ECM 830, San Diego, Calif.).

Briefly, 4×10⁵ cells in 0,4 gap cuvettes were subjected to 5 cycles of 20V, 10 ms in electroporation buffer pH 7.6 (120 mM KCl, 0,15 mM CaCl2, 10 mM K2HPO4/KH2PO4, 25 mM HEPES, 2 mM EGTA, 5 mM MgCl2, 50 mM Glutathion, 2 mM ATP).

Lack of Non-Specific Effect of siRNAs

Electroporation of DC did not induce significant toxicity in the cells neither after transfer of scramble or p50 siRNAs. Less than 10% of the cells were dead as measured by Trypan blue exclusion in 7 experiments.

Since DC are particularly apt at recognizing pathogen motifs such as double stranded RNA via the expression of specific Toll-like receptors, it was first checked whether or not a type-I interferon response was induced after transfection of siRNA in DC.

Human Interferon α levels were determined using specific ELISA kit (Biosource International, Camarillo, Calif.). The lower limit of detection was 25 pg/ml.

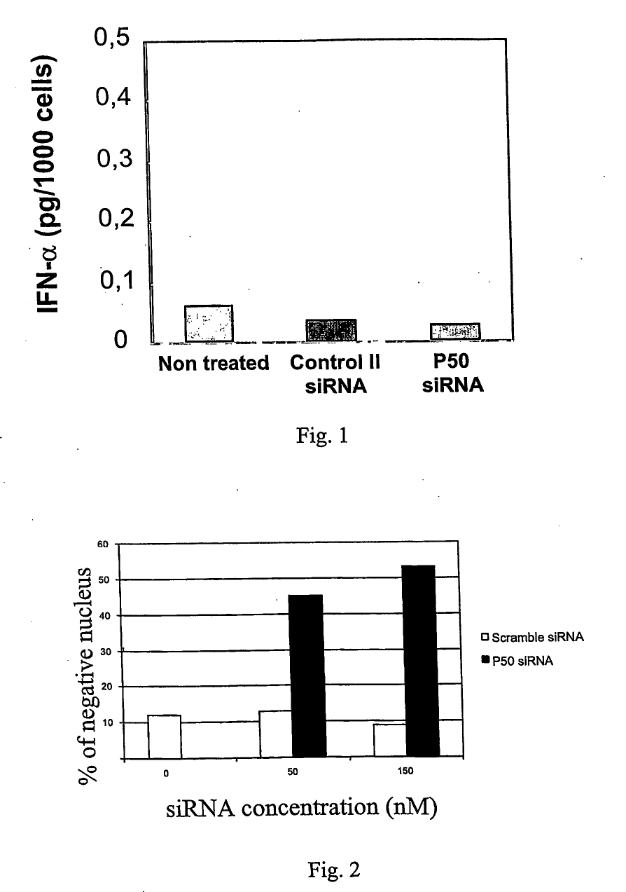

The results are shown on FIG. 1. Furthermore, supernatant fluids from DC cultures that were transfected with siRNAs were added to cultures of WISH fibroblasts infected with vesicular stomatatis virus and did not prevent the virus-induced lysis of WISH cells. This bio-assay further confirms the lack of type-I interferon production in culture medium after siRNA transfection of DC.

These results show that neither control siRNA nor p50 siRNA induces detectable type-1 IFN production.

Down-Regulation of p50

48 hours after electroporation with varying doses (1, 10, 50, 100, or 150 nM) of scramble or p50siRNA, expression of p50 in DC was evaluated by immuno-fluorescence.

0.5-1×10⁵ dendritic cells were spun on coverslips and fixed with 4% paraformaldehyde during 10 min at 4° C. Cells were washed twice in PBS then permeabilized in saponin buffer (0.1% saponin, 0.2% BSA, 0.02% sodium azide, in PBS). Non-specific Fc binding was blocked by incubation for 10 min. on ice with excess human gamma-globulin (1 mg/ml) and 1/100 dilution of donkey serum (Sigma, Saint Quentin Fallavier, France). Polyclonal goat antibodies specific for NFkB p50 (Sc-1191) (Santa Cruz Biotechnologies, Santa Cruz, Calif.) were used at 5 μg/ml followed by a FITC conjugated donkey anti-goat secondary reagent (Jackson Immunoresearch, West Grove, Pa.) used at 1/400 dilution in saponin buffer. Cells were observed under epifluorescence microscopy.

A dose-dependent extinction of p50 is particularly visible in the nucleus of DC with as little as 10 nM of p50 siRNA.

Results, expressed as percent of nucleated cells in the preparation whose nuclei show a dose-dependent extinction of p50 after electroporation with varying doses of control or p50 siRNA, are shown on FIG. 2. A significant down-regulation of p50 was obtained with 50 nM of p50 siRNA. The extinction was optimal with 100 nM siRNA (overall approximately 50% extinction; data not shown). Electroporation with 150 nM siRNA did not induce a significant increase of the extinction.

In order to confirm these results, the expression of the p50 protein and the p50 mRNA in DC electroporated with 100 nM of scramble or p50siRNA were respectively analyzed by Western blot and RT-PCR. Western blot.

After electroporation, 5×10⁵ cells were spun, resuspended in lysis buffer (50 mM tris, 150 mM NaCl, 1% TritonX100, 1% sodium Deoxicholate, 0.1% SDS, 5 mM EDTA, protease inhibitor cocktail) and kept at −80° C. until used. Equal amounts of protein (10 ug as determined by Bio-Rad DC Protein Assay, Bio-Rad, Hercules, Calif.) were separated on 10% polyacrylamide gels and transfert to nitrocellulose sheets. Polyclonal goat antibodies specific of p50 (Sc-1191) were used at 1/100 dilution. Anti-β actine (Sigma) was used as internal control. Horse Peroxidase conjugated rabbit anti-goat was used as secondary reagents at 1/5000 dilution. Standart immunostainings were carried out using the ECL Western Blotting Analysis System (Amersham Pharmacia, Buckinghamshire, England).

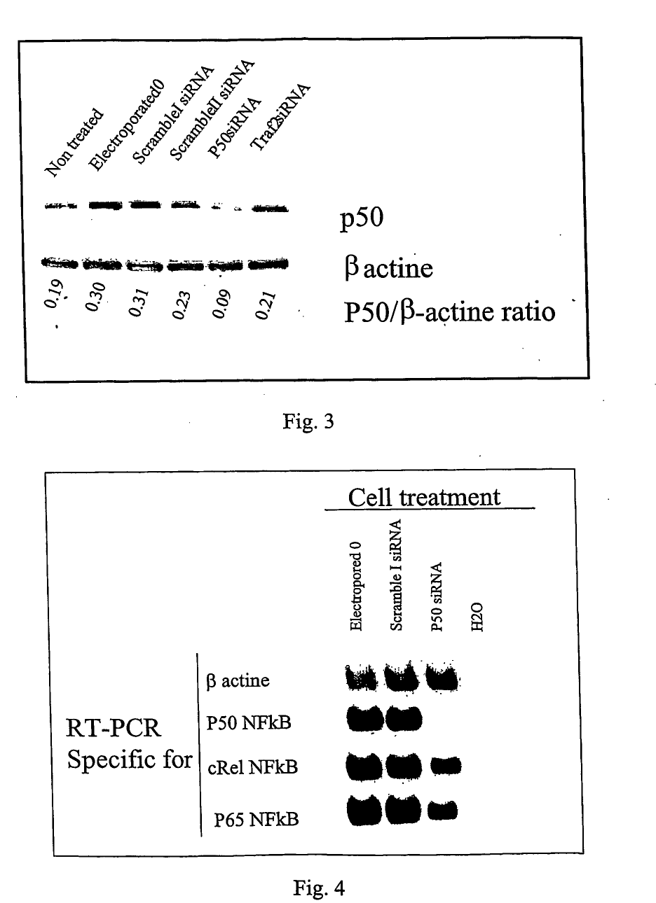

The results are shown on FIG. 3. Levels of p50 are specifically reduced by about half in DC transfected by p50 siRNA but not in untreated cells or in cells transfected with control or irrelevant siRNAs. RT-PCR analysis.

DC were electroporated with controls or p50 siRNA. After 24 hours, total cytoplasmic RNA was extracted from 5×10⁵ sorted cells using TRIzol reagent (all reagents from Gibco-InVitrogen, Cergy Pontoise, France). RT-PCR was done to analyze expression of p50, c-Rel, p65 and β-actine genes. PCR products were analyzed on 2% agarose gel electrophoresis stained with ethidium bromide.

The results are shown on FIG. 4. These results indicate that the reduction in p50 protein expression is due to a strong and specific down-regulation of p50 mRNA levels.

Reduction of IL-12 Production by p50 siRNA.

Immature DC were transfected with anti-P50 or scramble siRNA. 48 h after transfection, cells were harvested and washed twice in cytokine-free medium, prior to incubation with human recombinant CD40L trimer (1 μg/ml; Immunex), IL-1β (10 ng/ml R&D Systems). After overnight activation, supernatants were harvested and tested for IL-12 p70 by ELISA, using the OptEIA ELISA set for IL-12p70, according to manufacturer's instructions (BD-PharMingen). The lower limit of detection was 4 pg/ml.

The results of 3 independent experimentations are shown in table I below.

TABLE I

| | Condition | IL-12 p70 (pg/1000 cells) |
|---|---|---|
| Exp #1 | Non treated | 0.45 |
| | Scramble I siRNA | 0.29 |
| | P50siRNA | 0.015 |
| Exp #2 | Scramble II siRNA | 0.447 |
| | P50 siRNA | 0.148 |
| Exp #3 | Scramble II siRNA | 0.32 |
| | P50 siRNA | 0.058 |

These results show that treatment of DC with a siRNA anti p50 prior to activation with CD40L+IL-1 reproducibly and strongly reduces the secretion of IL-12.

Effect of p50 and cRel siRNAs on DC Phenotypic Maturation

Mature DC acquire expression of CD83, high levels of costimulatory antigens CD80 and CD86 and MHC class II molecules. To analyze the biological consequences of p50 reduction in monocyte-derived DC, expression of cell surface markers after stimulation with CD40L+IL-1β was measured by flow cytometric analysis on DC untreated or treated with 150 nM of scramble siRNA or p50, cRel or p50+cRel siRNAs.

Stainings of surface molecules were performed with the following antibodies: FITC conjugated mouse anti-human CDla, HLA-DR, PE conjugated mouse anti-human CD80, anti-CD83, APC-conjugated mouse anti-human HLA-DR, CD86. Cells were analyzed on a FACSCalibur instrument (Becton Dickinson) and data were analyzed using WinMDI (Version 2.8) software.

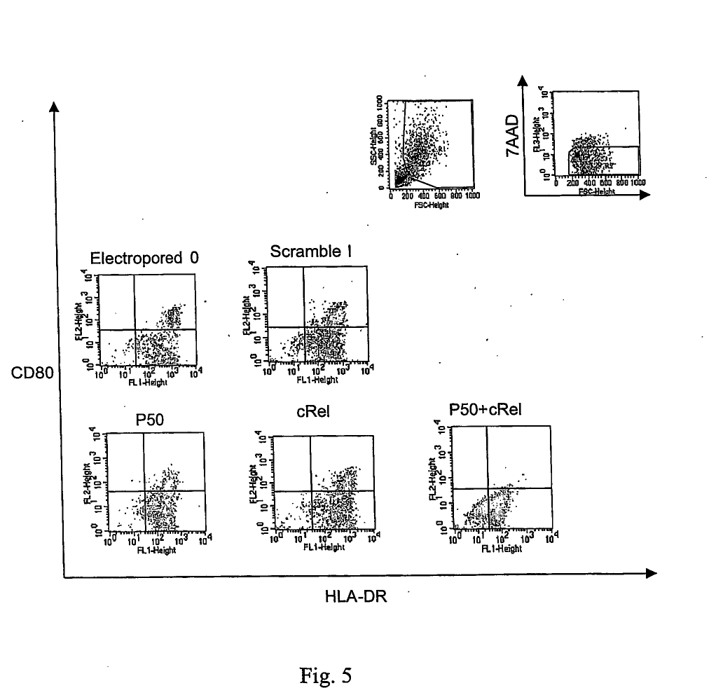

It was observed that treatment with scramble, p50, or cRel siRNAs induced no significant alteration in the expression of the maturation marker CD83 or of co-stimulatory molecules CD80, CD86, CD40 or MHC class II antigens (FIG. 5). However, combination of p50 and c-Rel siRNAs had a profound effect and reduced expression of HLA-DR, CD80 and CD86 on the cells with little effect on CD83 expression.

The results of treatments with p50, cRel or p50+cRel siRNAs on the expression of HLA-DR and CD80 markers are shown on FIG. 5.

Effect of p50 siRNA on T Cell Stimulating Properties of DC

Monocyte-derived DC have strong T cell stimulating properties and amounts as low as 1-10% of cells in a T cell culture are known to induce T cell proliferation and secretion of IFN-γ.

A mixed leukocyte reaction (MLR) was used to test the immunologic properties of DC transfected with p50 siRNA.

Purified T cells were prepared from cord blood mononuclear cells (MNC) using negative selection. MNC were incubated with human γ globulins (1 mg/ml) to block non-specific Fc receptor binding, then with monoclonal antibodies (mAbs) purified from hybridomas obtained from ATCC (Manassas, Va.) and specific for glycophorin A (10F7MN), CD14 (3C10-1E12), CD32 (IV3), CD11b (OKM1) and CD40 (G28-5). Red blood cells, phagocytes, B cells, monocytes and CD4⁺ T cells were then removed using magnetic beads coupled to goat anti-mouse antibodies (Dynal Inc., Lake Success, N.Y.). Magnetic bead selection was repeated after adding purified anti-CD20 and anti-HLA-DR antibodies (Caltag, Burlingame, Calif.) to further remove B cell and APCs. The negative fraction routinely contained >95% CD3⁺ T cells.

Allogeneic proliferation was performed by culturing for five days purified naive T cells (5×10⁴ cells per 0.2 ml of complete media per well in triplicate) with allogeneic 30 h-transfected DC. During the last 10 hours of culture, 1 μCi of (3H) thymidine (NEN, Boston, Mass.) was added to each well. Cells were harvested (Skatron Instruments, Maurepas, France) and counted using a liquid scintillation counter. Results are expressed as cpm±SD of triplicate wells.

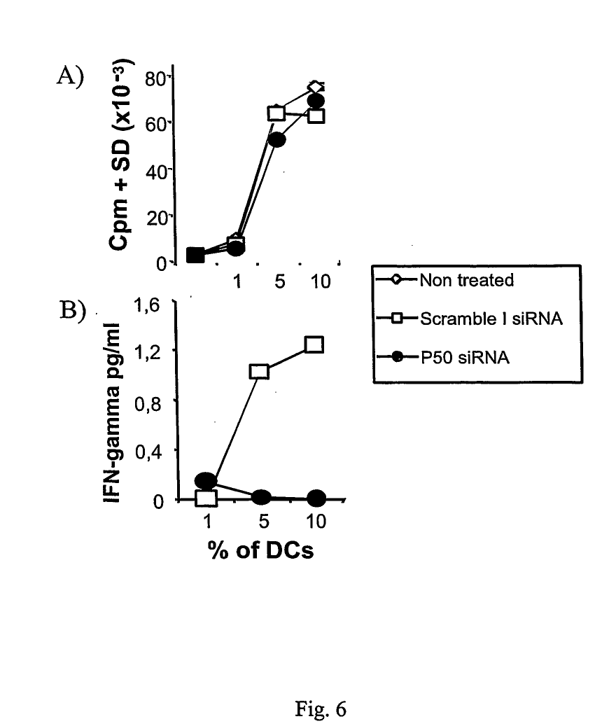

The results are shown in FIG. 6 A.

These results show that similar T cell proliferation is induced by non treated DC (⋄), DC treated with p50 siRNA (◯) or DC treated with scramble I siRNA (□).

Interferon gamma (IFNγ) is a cytokine resulting from a Th1 polarization of the immune response. It is produced by NK and T cells and it participates in the amplification of the immune response. In order to study qualitative aspects of the allogeneic response elicited, the production of IFNγ in the supernatants of the MLR was tested.

IFN-γ was measured using the OptEIA ELISA set for IFN-γ according to manufacturer's instructions (BD-PharMingen). The lower limit of detection was 4 pg/ml.

The results are shown in FIG. 6 B. A strong reduction in IFN-γ production is observed in cultures stimulated with DC treated with p50 siRNA (◯) when compared to non treated DC (⋄) or DC treated with scramble I siRNA (□).

### EXAMPLE 2

### EFFECT OF siRNA TARGETING TRAF PROTEINS IN DENDRITIC CELLS

siRNAs targeting TRAF3 (GUG CCA CCU GGU GCU GUG CdTdT; SEQ ID NO:5) and TRAF2 (GAA UAC GAG AGC UGC CAC GdTdT; SEQ ID NO:6) were designed from the sequences of the corresponding genes.

The sequences indicated above are the sense sequences of the siRNAs. The sequence for TRAF3 as well as the sequence for TRAF2 failed to reveal significant sequence homologies with other known genes (including other members of the same families) after standard BLAST search. Control scramble RNAs were also prepared, as described in Example 1.

Immature human monocyte-derived DC cells were transfected by electroporation with 150 nM of TRAF3 or TRAF2 siRNA, as described in Example 1.

Transfected DC were tested for their capacity to produce IL-12 upon CD40L+IL-1 activation, as described in Example 1.

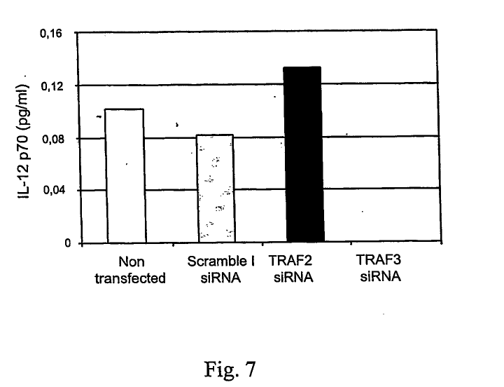

The results are shown in FIG. 7. While TRAF2 siRNA did not produce significant effects, TRAF3 siRNA significantly reduced the IL-12p70 production upon activation of DC.

A mixed leukocyte reaction (MLR) was used to test the immunologic properties of DC transfected with TRAF3 siRNA. T cell activation and IFNγ production were measured as as described in Example 1.

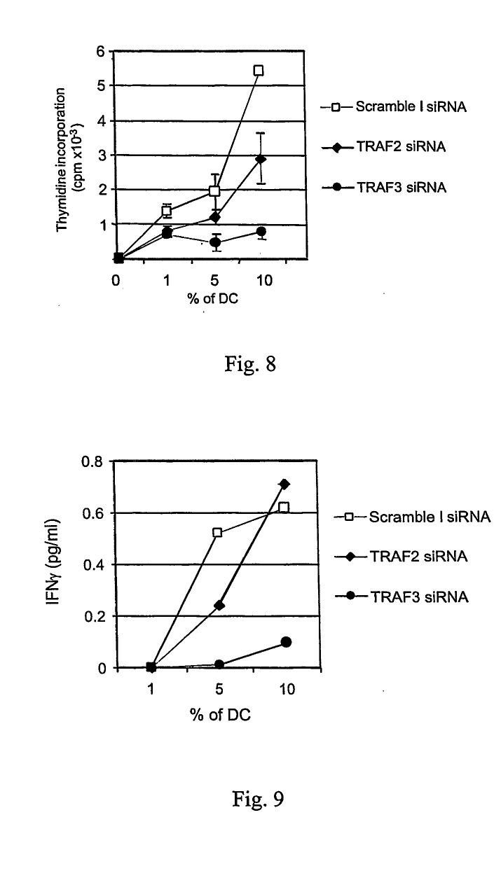

As shown in FIG. 8, an important reduction in T cell proliferation was observed when DC were transfected with TRAF3 siRNA (◯). Only at very high ratios of DC a little effect was observed with TRAF2 siRNA transfected-DC (⋄) when compared to DC transfected with scramble I siRNA (□).

FIG. 9 shows that there is an important reduction in the production of IFNγ by T cells stimulated with TRAF3 siRNA transfected-DC (◯), when compared with DC transfected with TRAF2 siRNA (⋄) or scramble I (□) siRNAs.

### EXAMPLE 3

### CONSTRUCTION OF AN EXPRESSION VECTOR FOR A p50 siRNA

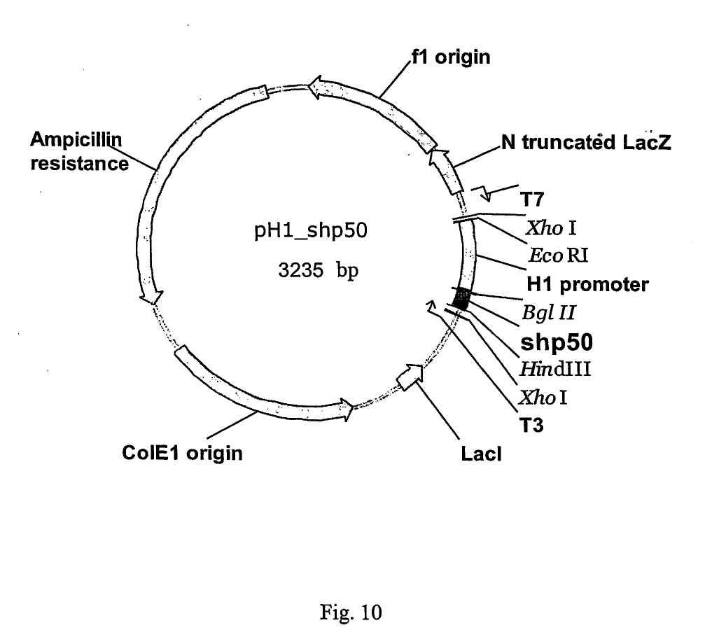

A plasmid comprising a DNA template for a p50 siRNA of SEQ ID NO:1 was constructed according to BRUMMELKAMP et al., (Science, 2002, cited above). This plasmid comprise a hairpin consisting of the DNA corresponding to the sense and antisense sequences of siRNA, separated by a spacer loop. This hairpin is placed under transcriptional control of the polIII promoter H1. Briefly, a sequence coding for the H1 promoter was obtained by PCR from genomic DNA of human peripheral blood mononuclear cells. This sequence was cloned into the EcoRI/HindIII site of the pBluescript phagemid vector. A XhoI restriction site was created by directed mutagenesis in position 5′ adjacent to the EcoR1 site, to obtain the pH1 plasmid. A BglII adapter sequence followed by the p50 hairpin and by a HindIII adapter was cloned into the BglII/HindIII site of the pH1 plasmid to obtain the pH1-shp50 vector, schematized on FIG. 10.

The sequence of the region of interest between XhoI sites in this pH1-shp50-1 plasmid is as follows

(SEQ ID NO:7): [symbol]
GTCGACGGTATCGATAAGCTTTTCCAAAAAGGGGCTATAATCCT
GGACTTCTCTTGAAAGTCCAGGATTATAGCCCCGGGGATCTGTGGTCTCA
TACAGAACTTATAAGATTCCCAAATCCAAAGACATTTCACGTTTATGGTG
ATTTCCCAGAACACATAGCGACATGCAAATATTGCAGGGCGCCACTCCCC
TGTCCCTCACAGCCATCTTCCTGCCAGGGCGCACGCGCGCTGGGTGTTCC
CGCCTAGTGACACTGGGCCCGCGATTCCTTGGAGCGGGTTGATGACGTCA
GCGTTCGAATTCCTGCAG

[symbol]

Letters in bold indicate the P50 small hairpin sequence; letters underlined indicate the H1 promoter and letters in bold and italic indicate the XhoI cloning site.

1. A method for obtaining isolated or cultured antigen presenting cells wherein the expression of one or more target gene(s) is down-regulated, wherein said method comprises introducing in said cells siRNA(s) directed against said target gene(s).

2. The method of claim 1, wherein said antigen presenting cells are dendritic cells or precursors thereof.

3. The method of claim 1, wherein at least one of said target gene(s) is selected from the group consisting of:
a gene encoding the p50 subunit of NF-κB;
a gene encoding TNF-receptor associated factor 3; and
a gene encoding the c-Rel subunit of NF-κB.

4. A siRNA directed against a target gene selected from the group consisting of:
a gene encoding the p50 subunit of NF-κB;
a gene encoding TNF-receptor associated factor 3; and
a gene encoding the c-Rel subunit of NF-κB.

5. An expression vector containing a DNA template for a siRNA of claim 4.

6. An immunosuppressive therapeutic composition comprising a siRNA of claim 4.

7. An antigen-presenting cell obtainable by the method of claim 1.

8. A pharmaceutical composition comprising an antigen-presenting cell of claim 7.

9. A method to produce T lymphocytes that fail to produce IFN-gamma, wherein said method comprises inducing activation of naïve T cells by co-cultivating said T cells with antigen presenting cells of claim 7, containing siRNA directed against a gene encoding p50.

10. A pharmaceutical composition comprising activated T lymphocytes obtainable by the method of claim 1.

11. An immunosuppressive therapeutic composition comprising an expression vector of claim 5.


SEQUENCE LISTING (7 sequences)

SEQ ID NO: 1 - RNA, 19 nt, Artificial Sequence
  p50 siRNA sense strand
ggggcuauaauccuggacu

SEQ ID NO: 2 - RNA, 19 nt, Artificial Sequence
  cRel siRNA sense strand
caaccgaacauacccuucu

SEQ ID NO: 3 - RNA, 19 nt, Artificial Sequence
  scramble I siRNA sense strand
uguuuuaagggccccccgu

SEQ ID NO: 4 - RNA, 19 nt, Artificial Sequence
  scramble II siRNA sense strand
cggcagcuagcgacgccau

SEQ ID NO: 5 - RNA, 19 nt, Artificial Sequence
  TRAF3 siRNA sense strand
gugccaccuggugcugugc

SEQ ID NO: 6 - RNA, 19 nt, Artificial Sequence
  TRAF2 siRNA sense strand
gaauacgagagcugccacg

SEQ ID NO: 7 - DNA, 324 bp, Artificial Sequence
  Expression cassette for p50 siRNA
ctcgaggtcgacggtatcgataagcttttccaaaaaggggctataatcctggacttctcttgaaagtccaggattatagccccggggatctgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaattcctgcagctcgag